(19) United States
(12) Patent Application Publication
(10) Pub. No.: US 2022/0378302 A1
(43) Pub. Date: Dec. 1, 2022

(54) SYSTEMS, METHODS, AND VEHICLES FOR PASSENGER TRANSPORTATION AND HEALTH MONITORING

(71) Applicant: Toyota Motor Engineering & Manufacturing North America, Inc., Plano, TX (US)

(72) Inventors: Frederico Marcolino Quintao Severgnini, Ann Arbor, MI (US); Ercan Mehmet Dede, Ann Arbor, MI (US); Chungchih Chou, Ann Arbor, MI (US)

(73) Assignee: Toyota Motor Engineering & Manufacturing North America, Inc., Plano, TX (US)

(21) Appl. No.: 17/335,696

(22) Filed: Jun. 1, 2021

(51) Int. Cl. A61B 5/0205; A61B 5/18; A61B 5/00

(52) U.S. Cl. CPC A61B 5/02055; A61B 5/18; A61B 5/0006; A61B 5/0008; A61B 5/0013; A61B 5/0022; A61B 2562/0271

(57) ABSTRACT

A method for passenger transportation and health monitoring includes determining, with a contactless biomonitoring sensor, a location of one or more passengers within a monitoring area of a vehicle, and generating, with the contactless biomonitoring sensor, a health profile of one or more passengers. The method may also include identifying, with a processor, a health condition, an action, an accompanying item, or combinations thereof of one or more passengers based on the health profile corresponding to the one or more passengers, and determining, with the processor, a vehicle action based on the health condition, the action, the accompanying item, or combinations thereof. The method may further include directing, with the processor, the vehicle to perform the vehicle action.

### TECHNICAL FIELD

The present disclosure relates to passenger transportation and health monitoring, and more specifically to systems, methods, and vehicles for passenger transportation and health monitoring using contactless biomonitoring sensors.

### BACKGROUND

Autonomous vehicles typically do not contain a driver and thus may not have someone available to monitor and respond when a passenger transportation or health issue arises. Vehicles may have additional health sensors to monitor for health issues. However, having sensors for health monitoring as well as sensors for autonomous driving can increase the cost of building and maintaining the vehicle. For example, the vehicle may require a LIDAR sensor to drive and may also require multiple electrodes to monitor a passenger's heart activity. The added sensors add costs to manufacturing the vehicle as well as add new points of potential need for repair. Additionally, health sensors generally require direct physical contact with the passenger. For example, a seatbelt respiration monitor cannot monitor a passenger's respiration rate unless the passenger has fastened the seatbelt, and a seat-based heart activity monitor cannot monitor a passenger's heart activity unless the passenger is seated.

Therefore, a need exists for vehicle passenger transportation and health monitoring with fewer sensors that can monitor passengers without maintaining direct physical contact.

### SUMMARY

In one embodiment, a method for passenger transportation and health monitoring includes determining, with a contactless biomonitoring sensor, a location of one or more passengers within a monitoring area of a vehicle, and generating, with the contactless biomonitoring sensor, a health profile of one or more passengers. The method may also include identifying, with a processor, a health condition, an action, an accompanying item, or combinations thereof of one or more passengers based on the health profile corresponding to the one or more passengers, and determining, with the processor, a vehicle action based on the health condition, the action, the accompanying item, or combinations thereof. The method may further include directing, with the processor, the vehicle to perform the vehicle action.

In another embodiment, a system for passenger transportation and health monitoring includes a processor, a contactless biomonitoring sensor communicatively coupled to the processor, a memory module communicatively coupled to the processor, and machine-readable instructions stored in the memory module. The machine-readable instructions, when executed by the processor, may cause the processor to determine, with the contactless biomonitoring sensor, a location of one or more passengers within a monitoring area of a vehicle, and generate, with the contactless biomonitoring sensor, a health profile of one or more passengers. The machine-readable instructions may also cause the processor to identify, with the processor, a health condition, an action, an accompanying item, or combinations thereof of one or more passengers based on the health profile corresponding to the one or more passengers, and determine, with the processor, a vehicle action based on the health condition, the action, the accompanying item, or combinations thereof. The machine-readable instructions may further cause the processor to direct, with the processor, the vehicle to perform the vehicle action.

In yet another embodiment, a vehicle for passenger transportation and health monitoring includes a processor, a contactless biomonitoring sensor communicatively coupled to the processor, a memory module communicatively coupled to the processor, and machine-readable instructions stored in the memory module. The machine-readable instructions, when executed by the processor, may cause the processor to determine, with the contactless biomonitoring sensor, a location of one or more passengers within a monitoring area of the vehicle, and generate, with the contactless biomonitoring sensor, a health profile of one or more passengers. The machine-readable instructions may also cause the processor to identify, with the processor, a health condition, an action, an accompanying item, or combinations thereof of one or more passengers based on the health profile corresponding to the one or more passengers, and determine, with the processor, a vehicle action based on the health condition, the action, the accompanying item, or combinations thereof. The machine-readable instructions may further cause the processor to direct, with the processor, the vehicle to perform the vehicle action.

These and additional features provided by the embodiments of the present disclosure will be more fully understood in view of the following detailed description, in conjunction with the drawings.

### BRIEF DESCRIPTION OF THE DRAWINGS

The following detailed description of specific embodiments of the present disclosure can be best understood when read in conjunction with the following drawings, where like structure is indicated with like reference numerals and in which:

FIG. 1 depicts a system for passenger transportation and health monitoring, according to one or more embodiments shown and described herein;

FIG. 2 schematically depicts internal hardware components of the system of FIG. 1, according to one or more embodiments shown and described herein;

FIG. 3 depicts a flowchart of a method for passenger transportation and health monitoring, according to one or more embodiments shown and described herein;

FIG. 4 depicts a passenger inside of a vehicle for transportation and health monitoring, according to one or more embodiments shown and described herein; and

FIG. 5 depicts a passenger outside of a vehicle for transportation and health monitoring, according to one or more embodiments shown and described herein.

### DETAILED DESCRIPTION

Biometrics are body measurements and calculations related to human characteristics, which may be used to label and describe individuals. In embodiments of the present disclosure, an individual's gait may be biometric data that demonstrates a unique pattern of behavior that may identify the individual.

Vital signs are measurements used to assess the general physical health of an individual. Vital signs include measurements such as body temperature, heart rate, respiration rate, and blood pressure.

Biomonitoring, for purposes of the present disclosure, is the monitoring of biometrics and/or vital signs. Typical sensors for biomonitoring require the sensor to come into contact with the user. For example, gait sensors may require the user to walk on a pressure-sensitive pad or wear inertial measurement units (IMU) sensors, and heart rate monitors may require the user to wear/hold electrodes.

Contactless biomonitoring sensors perform biomonitoring via contactless sensors, such as LIDAR and RADAR. The use of contactless biomonitoring sensors allows the system to monitor the health of passengers throughout the vehicle and is not limited to areas where the passenger can be in contact with the sensor. Additionally, the use of contactless biomonitoring sensors allows the system to monitor the health of more than one passenger at a time.

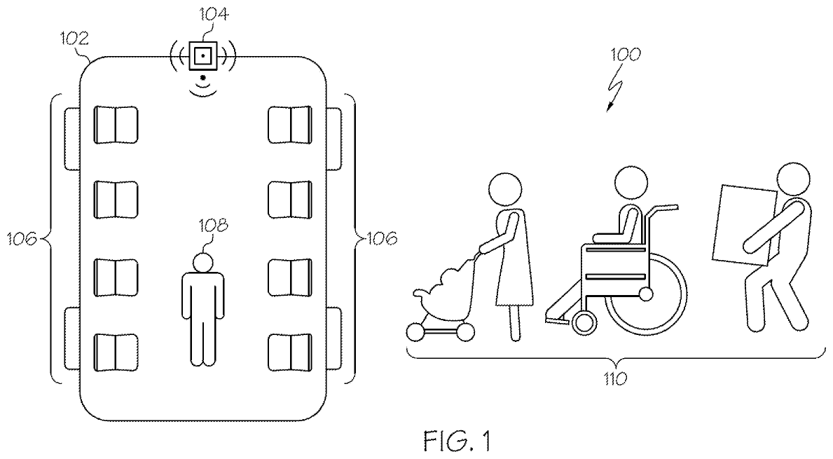

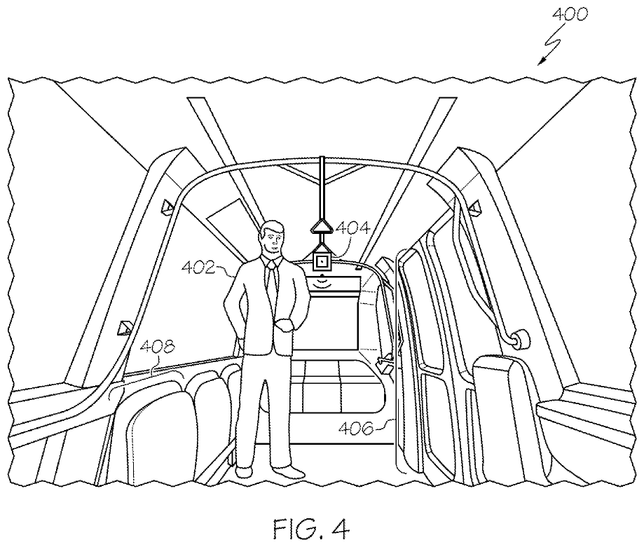

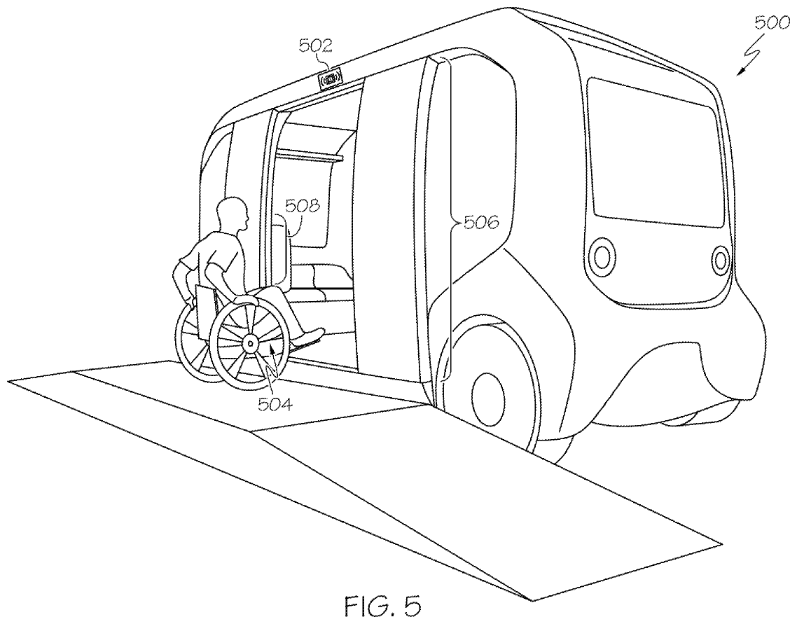

Embodiments of the present disclosure are directed to passenger transportation and health monitoring. Health monitoring may include the use of contactless biomonitoring sensors to determine one or more passenger's biometrics and/or vital signs. Embodiments may use contactless biomonitoring sensors to determine the location of one or more passengers and identify their health conditions, actions, and/or accompanying items, as shown in FIG. 1. Embodiments of the present disclosure may include contactless biomonitoring sensors on the interior of the vehicle, as illustrated in FIG. 4, and/or may include contactless biomonitoring sensors on the exterior of the vehicle, as illustrated in FIG. 5.

Accordingly, disclosed herein are systems, methods, and vehicles for passenger transportation and health monitoring.

Referring initially to FIG. 1, a system 100 for passenger transportation and health monitoring is depicted. The system 100 may include a vehicle 102, a contactless biomonitoring sensor 104, a set of seats 106, a passenger 108, and a set of passengers needing accommodations 110. In FIG. 1, only a single vehicle 102, contactless biomonitoring sensor 104, passenger 108 is shown; however, it should be understood that any number of items depicted in FIG. 1 may be included. The system 100 may be used to carry out the methods as described herein.

The vehicle 102 may be a car, a bus, a van, or any other vehicle that can transport one or more passengers. The vehicle 102 may be manually driven and/or autonomous. Additionally, the vehicle 102 may be a connected vehicle, meaning the vehicle 102 may establish wireless connectivity with a remote server and/or with other connected vehicles. The vehicle 102 may also contain seating for passengers 108, 110, as well as compartments for the storage of items accompanying the passengers 108, 110.

The vehicle 102 is equipped with one or more contactless biomonitoring sensors 104 to enable it to carry out the methods as discussed herein. The contactless biomonitoring sensors 104 may determine a passenger's location, health condition, and/or accompanying items. This information may be utilized to help the vehicle 102 take an action in response to the determined location, health condition, and/or accompanying items.

Contactless biomonitoring sensors 104 refer to sensors that may be used to measure biometrics and/or vital signs without contact with a user and can be used to measure multiple users at once. Contactless biomonitoring sensors 104 utilize contactless sensors such as, for example, LIDAR, RADAR, ultrasound, infrared (IR), imaging (e.g., cameras), and EEG-based sensors. Contactless biomonitoring sensors 104 may be affixed to the vehicle 102 and may be located inside and/or outside of the vehicle 102.

Regarding biometrics, contactless biomonitoring sensors 104 may, for example, detect gait patterns, facial features, and brainwaves. LIDAR and RADAR, among other contactless sensors, may analyze gait patterns because these sensors allow for the continuous acquisition of distance data as the user walks. Neurobiomonitoring (NBM) coils are an EEG-based sensor that may detect brainwaves emitted by the user and may be used in combination with an interface (e.g., a screen) to present the user with a stimulus (e.g., an image) to invoke a certain brainwave response unique to the user.

Regarding vital signs, contactless biomonitoring sensors 104 may, for example, detect respiration rate, heart activity, body temperature, and blood oxygenation.

LIDAR, RADAR, and ultrasound, among other contactless sensors, may measure respiration rate by comparing the difference in distance between the user's chest when inhaling and exhaling over a period of time and/or by measuring the refraction of signals displaced by a user's chest movement between inhaling and exhaling.

For example, when a user sits facing a sensor and inhales, the user's chest expands and gets closer to the sensor; but when the user sits facing a sensor and exhales, the user's chest contracts and gets further from the sensor. Plotting on a graph the distance the signal of the sensor travels between the sensor and the user would form a wave, the phase of the wave being related to the distance traveled by the signal.

Additionally, LIDAR, RADAR, and ultrasound, among other contactless sensors, may measure heart activity, including heart rate and heart rate variability. Heart activity may be monitored by measuring the slight variations in the phase of the respiration measurement. A user's heartbeats cause slight movements of different parts of the user's body. The slight movements translate to slight fluctuations in the breathing motion that may be detected by a contactless sensor. The average number of heartbeats over a period of time may be interpreted as the user's heart rate. The delay between successive heartbeats may be interpreted as the user's heart rate variability.

For example, when a user sits facing a sensor and inhales, the user's chest expands and gets closer to the sensor; but when the user sits facing a sensor and exhales, the user's chest contracts and gets further from the sensor. The slight fluctuations in the breathing motion caused by heartbeats may be reflected in the distance measured by the sensor.

Furthermore, cameras, among other contactless sensors, may measure body temperature and/or blood oxygenation. For example, infrared thermal cameras may monitor body temperature. Body temperature may be monitored by measuring the infrared radiation of the user. As another example, red, green, and blue cameras (RGB cameras) may monitor blood oxygenation. Blood oxygenation may be monitored by measuring the color variations of a user's skin.

It should be understood that contactless biomonitoring sensors 104 are not limited to LIDAR. RADAR, ultrasound, and cameras. Embodiments are also not limited to the specific methods described for determining respiration rate, heart activity, body temperature, and blood oxygenation. Additionally, contactless biomonitoring sensors 104 are not limited to measuring only respiration rate, heart activity, body temperature, and blood oxygenation. Furthermore, the vehicle 102 may use one or more contactless biomonitoring sensors 104, independently or in combination, to carry out the embodiments as described herein.

Still referring to FIG. 1, the vehicle 102 may include an adjustable set of seats 106. Adjusting a seat 106 may include deploying or retracting the seat. As an example of the type of accommodations a vehicle 102 may offer, the vehicle 102 may deploy an appropriate number of seats according to the number of passengers detected by the contactless biomonitoring sensor 104. For example, if the contactless biomonitoring sensors 104 of the vehicle 102 detect four passengers, the vehicle 102 may deploy four seats 106. When the seats 106 are already deployed, the vehicle 102 may retract an appropriate number of seats according to a health condition and/or a passenger's accompanying item. For example, if the contactless biomonitoring sensors 104 of the vehicle 102 detect a passenger in a wheelchair the vehicle 102 may retract seats 106 such that the passenger in a wheelchair may stay to the side of the vehicle 102 interior and make way for other passengers to pass through.

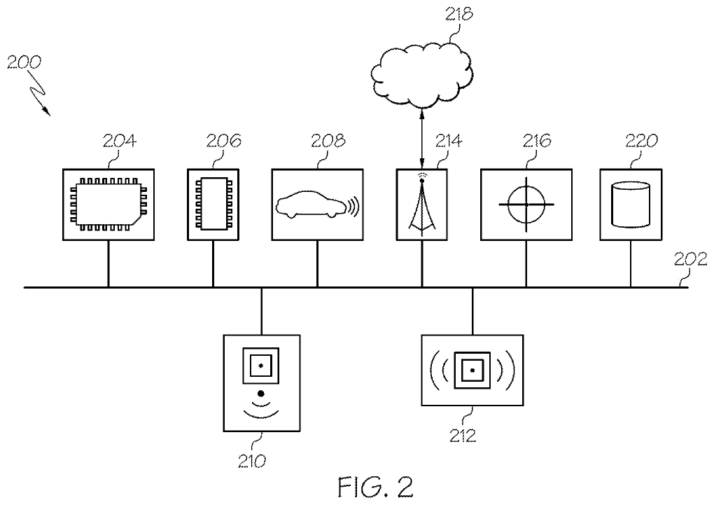

Referring now to FIG. 2, internal hardware components of the vehicle 200 of FIG. 1 are schematically depicted. The vehicle 200 may include a processor component 204, a memory component 206, a driving sensor component 208, a network connectivity component 214, a satellite component 216, a data storage component 220, an internal contactless biomonitoring sensor 210, and an external contactless biomonitoring sensor 212. The vehicle 200 may also include a communication path 202 that communicatively connects the foregoing components of the vehicle 200.

The processor component 204 may include one or more processors that may be any device capable of executing machine-readable and executable instructions. Accordingly, each of the one or more processors of the processor component 204 may be a controller, an integrated circuit, a microchip, or any other computing device. The processor component 204 is coupled to the communication path 202 that provides signal connectivity between the various components of the vehicle 200. Accordingly, the communication path 202 may communicatively couple any number of processors of the processor component 204 with one another and allow them to operate in a distributed computing environment. Specifically, each processor may operate as a node that may send and/or receive data. As used herein, the phrase “communicatively coupled” means that coupled components are capable of exchanging data signals with one another such as, e.g., electrical signals via a conductive medium, electromagnetic signals via air, optical signals via optical waveguides, and the like.

Accordingly, the communication path 202 may be formed from any medium that is capable of transmitting a signal such as, e.g., conductive wires, conductive traces, optical waveguides, and the like. In some embodiments, the communication path 202 may facilitate the transmission of wireless signals, such as Wi-Fi, Bluetooth, Near-Field Communication (NFC), and the like. Moreover, the communication path 202 may be formed from a combination of mediums capable of transmitting signals. In one embodiment, the communication path 202 comprises a combination of conductive traces, conductive wires, connectors, and buses that cooperate to permit the transmission of electrical data signals to components such as processors, memories, sensors, input devices, output devices, and communication devices. Accordingly, the communication path 202 may comprise a vehicle bus, such as for example a LIN bus, a CAN bus, a VAN bus, and the like. Additionally, it is noted that the term “signal” means a waveform (e.g., electrical, optical, magnetic, mechanical, or electromagnetic), such as DC, AC, sinusoidal-wave, triangular-wave, square-wave, vibration, and the like, capable of traveling through a medium.

The memory component 206 is coupled to the communication path 202 and may contain one or more memory modules comprising RAM. ROM, flash memories, hard drives, or any device capable of storing machine-readable and executable instructions such that the machine-readable and executable instructions can be accessed by the processor component 204. The machine-readable and executable instructions may comprise logic or algorithms written in any programming language of any generation (e.g., 1GL, 2GL, 3GL, 4GL, or 5GL) such as, e.g., machine language, that may be directly executed by the processor, or assembly language, object-oriented languages, scripting languages, microcode, and the like, that may be compiled or assembled into machine-readable and executable instructions. Alternatively, the machine-readable and executable instructions may be written in a hardware description language (HDL), such as logic implemented via either a field-programmable gate array (FPGA) configuration or an application-specific integrated circuit (ASIC), or their equivalents. Accordingly, the methods described herein may be implemented on any conventional computer programming language, as pre-programmed hardware elements, or as a combination of hardware and software components.

The driving sensor component 208 is coupled to the communication path 202 and is communicatively coupled to the processor component 204. The driving sensor component 208 may navigate or assist in the navigation of the vehicle 200. The driving sensor component 208 may also be used to perform various driving assistance including, but not limited to autonomous driving, advanced driver-assistance systems (ADAS), adaptive cruise control (ACC), cooperative adaptive cruise control (CACC), lane change assistance, anti-lock braking system (ABS), collision avoidance system, and the like. The driving sensor component 208 may include, e.g., LIDAR sensors. RADAR sensors, optical sensors, laser sensors, proximity sensors, location sensors, and the like to assist in the performing of the various driving assistance.

The network connectivity component 214 communicatively couples the vehicle 200 with a network 218. The network connectivity component 214 is coupled to the communication path 202 such that the communication path 202 communicatively couples the network connectivity component 214 to other components of the vehicle 200. The network connectivity component 214 can be any device capable of transmitting and/or receiving data via a wireless network. Accordingly, the network connectivity component 214 can comprise a communication transceiver for sending and/or receiving data according to any wireless communication standard. For example, the network connectivity component 214 can comprise a chipset (e.g., antenna, processors, machine-readable instructions, etc.) to communicate over wired and/or wireless computer networks such as, for example, Wi-Fi, WiMAX. Bluetooth, IrDA, Ethernet, Z-Wave, ZigBee, and the like.

The network 218 may comprise any wired and/or wireless network such as, for example, wide area networks, metropolitan area networks, local area networks, satellite networks, cellular networks, and the like. Additionally, it is noted that the vehicle 200 and any additional computing devices can share resources with one another over the network 218 such as, for example, via the wired portion of the network, the wireless portion of the network, or combinations thereof.

A satellite component 216 is coupled to the communication path 202 and communicatively coupled to the processor component 204. The satellite component 216 may comprise one or more antennas configured to receive signals from global positioning system (GPS) satellites. Specifically, in one embodiment, the satellite component 216 includes one or more conductive elements that interact with electromagnetic signals transmitted by global positioning system satellites. The received signal is transformed into a data signal indicative of the location (e.g., latitude and longitude) of the satellite component 216, and consequently, the vehicle 200.

The data storage component 220 may store data used by various components of the vehicle 200. The data storage component 220 may be included in the memory component 206. In addition, the data storage component 220 may store data gathered by the driving sensor component 208, the internal contactless biomonitoring sensor 210, and/or the external contactless biomonitoring sensor 212.

The internal contactless biomonitoring sensor 210 may be located inside the vehicle 200. The internal contactless biomonitoring sensors 210 may include sensors to monitor passenger health, such as RADAR sensors for monitoring heart activity and IR sensors for monitoring body temperature. The internal contactless biomonitoring sensors 210 may also determine passenger location so that the health data may be attributed to the appropriate passenger and so that the vehicle 200 may take an action in the appropriate part of the vehicle.

The external contactless biomonitoring sensor 212 may contain the same set of sensors as the internal contactless biomonitoring sensors 210 or may contain different sensors depending on its intended task. For example, the external contactless biomonitoring sensors 212 may include sensors, such as LIDAR and EEG-based sensors, to authenticate prospective passengers and determine their accompanying items. LIDAR sensors may be used to identify an object that a prospective passenger is carrying to determine whether to open a cargo area of the vehicle 200. EEG-based sensors may be used in combination with a display for presenting a visual stimulus to a passenger to invoke a brainwave response that may be detected by the EEG-based sensors to verify the identity of the passenger.

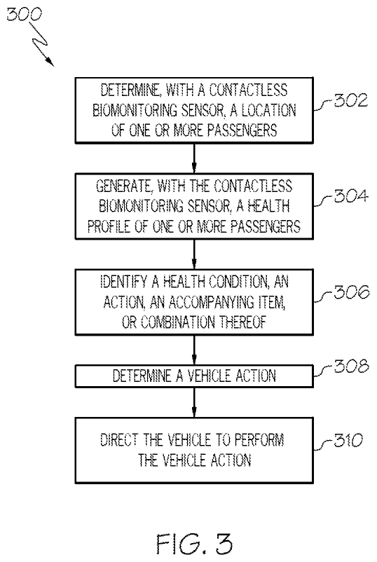

Referring now to FIG. 3, a method 300 for passenger transportation and health monitoring is depicted. The method 300 is described by referring to FIG. 1 and FIG. 2, discussed above. Embodiments of the methods described herein are not limited to the steps included in FIG. 3 nor are embodiments limited to the order as shown in FIG. 3.

At block 302, a location of passengers 108, 110 is determined with a contactless biomonitoring sensor 104. Although contactless biomonitoring sensors 104 as described herein may determine biometrics and/or vital signs of the passengers 108, 110, contactless biomonitoring sensors 104 are also able to determine the location of the passengers 108, 110. The location of passengers 108, 110 may include position, stance, orientation, and other spatial location information.

At block 304, a health profile is generated for each passenger 108, 110. Determining the location of the passengers 108, 110 may allow the vehicle 102 to maintain separate health profiles for each passenger 108, 110 so as not to confuse the health data of one passenger for that of another. The contactless biomonitoring sensors 104 may continuously monitor the location of the passengers 108, 110 so the passengers 108, 110 do not need to be in constant contact with a sensor for biomonitoring.

Health profiles may be generated by using the contactless biomonitoring sensors 104 to gather a set of health data from within the monitoring area of the vehicle. The monitoring area may be inside the vehicle and/or in a predetermined vicinity outside of the vehicle, such as outside of the doorway through which passengers would enter the vehicle. The monitoring area of FIG. 1, for example, is inside the vehicle 102 and outside of the vehicle 102 entrance. The health data may include biometric data and/or vital signs of the passengers. The health data may be segmented based on the location of each passenger and assigned to the passenger at the passenger's corresponding location.

At block 306, a health condition, an action, and/or an accompanying item of the passengers 108, 110 is identified.

A health condition includes any abnormal vital signs. For example, a health condition may include an elevated respiration rate, body temperature, and heart rate. Abnormal vital signs may be indicative of an underlying illness or infection. For example, if a passenger has a fever in conjunction with poor breathing, the passenger may have the flu. A health condition also includes any health emergency. A health emergency would be a medical event requiring immediate medical attention. For example, if the contactless biomonitoring sensor 104 detects that a passenger 108, 110 is not breathing, the passenger 108, 110 may be having a health emergency.

An action includes any movement or behavior of the passenger 108, 110. For example, a passenger 108, 110 moving around in the cabin of the vehicle 102 may be an action. A passenger 108, 110 in an aggressive stance and other aggressive body language may be an action indicating that the passenger 108, 110 is acting threateningly towards other passengers.

An accompanying item includes any item that a passenger may bring onto the vehicle 102. For example, a suitcase, a service animal, a wheelchair, and a stroller may be items that accompany a passenger 108, 110 as the passenger 108, 110 boards the vehicle 102.

At block 308, the vehicle 102 determines a vehicle action in response to the identified health condition, action, and/or accompanying item. A vehicle action is an action that can be performed by or with components of the vehicle 102. Vehicle actions may include, but are not limited to, the following example actions.

The vehicle 102 may adjust seats 106 by deploy or retracting one or more seats 106. For example, based on the number of passenger locations determined, an exact number of seats corresponding to the number of passengers in the vehicle 200 may be deployed at the locations in which the passengers are standing. If a passenger is holding a child, the vehicle 102 may detect the child, and a seat may be deployed for both the adult passenger and child passenger at their location in the vehicle 102. If a passenger requires the use of a mobility device, such as a wheelchair, the vehicle 102 may retract, rather than deploy, multiple seats to accommodate for the size of the mobility device.

As another example, the vehicle 102 may open a storage compartment of the vehicle 102. For example, if a passenger is carrying a large suitcase, it may be preferable to have the suitcase in a storage location to make room for the rest of the passengers. If LIDAR detects that the passenger is carrying an object, for instance by identifying the object or by identifying the passenger's gait as being that of someone who is carrying a suitcase, the vehicle 102 may open a storage compartment to receive the suitcase.

The vehicle 102 may prevent a passenger from boarding the vehicle 102. For example, if a passenger has a fever in conjunction with poor breathing, the passenger 108, 110 may have the flu. To prevent the flu from spreading to other passengers, vehicle 102 may keep its doors closed and direct the passenger to stay away from the vehicle 102. As another example, if the external contactless biomonitoring sensors 212 detect, via RADAR and/or LIDAR, that a passenger 108, 110 is concealing a harmful object and is acting threatening towards other passengers, the vehicle 102 may evacuate the vehicle and call emergency responders.

The vehicle 102 may also warn or generate instructions for the passengers. For example, if aggressive behavior from a passenger is identified, the vehicle 102 may stop, open its doors, and instruct the passengers 108, 110 to evacuate. The vehicle 102 may also call emergency responders if the passenger is being too aggressive. The vehicle 102 may also simply issue a warning to the passengers. For example, if the vehicle 102 determines that a passenger is moving around the passenger cabin while the vehicle 102 is in motion, the vehicle 102 may issue an audio warning such as, “please remain seated until the vehicle has come to a complete stop.”

The vehicle 102 may also navigate to a health center, communicate with an emergency responder, and/or adjust a comfort system of the vehicle. For example, if the contactless biomonitoring sensor 104 detects that a passenger 108, 110 is having trouble breathing, the passenger may be having a health emergency. If there is a health emergency, the vehicle 102 may contact an emergency responder to request an ambulance come to the vehicle 102 to help the passenger. The vehicle 102 may also adjust a comfort system, such as turning on the air conditioner or opening a window, to assist with the emergency situation. If the health emergency is severe, the vehicle 102 may navigate itself to the nearest health center.

At block 310, the vehicle 102 is directed to perform the determined vehicle action. In some embodiments, the vehicle 102 may have a driver who may confirm the determined vehicle action before the vehicle 102 performs the action.

Referring now to FIG. 4, a passenger inside of a vehicle for transportation and health monitoring is depicted. The vehicle 400 may be an autonomous rideshare or public transportation vehicle with an interior area having adjustable seats and doors. The seats are adjustable in that they may be deployed and retracted, and the doors are adjustable in that they may be opened and closed. The interior area may be monitored by an interior contactless biomonitoring sensor 404. The interior contactless biomonitoring sensors 404 may be one or more sensors including RADAR, LIDAR, ultrasound, IR, imaging, and the like. The contactless biomonitoring sensors 404 can determine how many passengers are in the vehicle 400 by detecting one or more different heart activities and/or respiration patterns.

As the passenger 402 first enters the vehicle 400 through the door 406, the vehicle 400 may recognize the location of a new passenger at the location of the door 406 and may first check for accompanying items. The contactless biomonitoring sensors 404 may determine that the passenger 402 has an accompanying item based on recognition of the shape of the object and/or recognition of the passenger's gait pattern that is indicative of a person holding an object. If an object is detected, the vehicle 400 may open a storage compartment for the passenger 402 to place the object.

Once the passenger 402 enters the vehicle 400 through the door 406, the contactless biomonitoring sensors 404 may begin determining the location of the passenger 402 throughout the passenger cabin. The vehicle 400 may track the passenger's location and orientation to determine which seat should be adjusted for the passenger 402. For example, as the passenger 402 engages in the action of approaching a seat 408, the vehicle 102 may automatically take an action of deploying the seat 408.

As the passenger 402 is riding the vehicle 400, the vehicle 400 may monitor the health status of the passenger 402. The vehicle 400 may continuously or periodically monitor the health status of the passenger 402 by determining the passenger's location and attempting to identify a health condition. Monitoring the location of the passenger 402 may help the contactless biomonitoring sensor 404 focus where it is sensing from as well as attributing the sensed health data to the passenger 402. The sensed health data of the passenger 402 may be continuously or periodically analyzed to identify a health condition of the passenger 402. If a health condition is identified, the vehicle 400 may be directed to perform a vehicle action in response to the determined location and the identified health condition. For example, if the contactless biomonitoring sensor 404 identifies that the passenger 402 is having trouble breathing, then the vehicle 400 may turn on the air conditioning.

As the vehicle 400 is approaching its next stop, the vehicle 400 may instruct the passenger 402 to remain seated and/or open the storage compartment if the passenger 402 had any accompanying items. The contactless biomonitoring sensors 404 may detect that the user is standing based on a sudden increase in heart rate, for example. The vehicle 400 may play an audio notice instructing the user to remain seated until the vehicle 400 comes to a stop. If the passenger 402 is oriented towards and approaching the storage compartment, the vehicle 400 may open the storage compartment for the passenger to retrieve the passenger's accompanying items.

As an additional example, if the passenger 402 is holding a child, the contactless biomonitoring sensors 404 may detect multiple respiration rates and deploy seats 408 for both the passenger 402 and the child. If the passenger 402 is in a wheelchair, the contactless biomonitoring sensors 404 may detect the presence of the wheelchair via a RADAR sensor, for example, and forego deploying a chair for this passenger 402. If the passenger has an accompanying service animal, the contactless biomonitoring sensors 404 may detect the presence of the animal via a RADAR sensor, for example, and retract seats to make room for the animal to sit. Additionally, if the passenger 402 is carrying a dangerous item, such as a concealed weapon, the contactless biomonitoring sensors 404 may detect the presence of the dangerous item via a RADAR sensor, for example, and prevent the passenger 402 from entering the vehicle 400 and/or contact emergency responders.

Although an autonomous rideshare or public transportation vehicle was referenced for purposes of illustration, any vehicle could implement embodiments of the present disclosure. For example, vehicles that are manually driven may include embodiments of the present disclosure, and drivers may be used to confirm the conditions that embodiments have detected before taking action.

Referring now to FIG. 5, a passenger 504 outside of a vehicle 500 for transportation and health monitoring is depicted. The vehicle 500 may be an autonomous rideshare or public transportation vehicle with an exterior area for passengers waiting to board the vehicle 500. The exterior area may be monitored by an exterior contactless biomonitoring sensor 502. The exterior contactless biomonitoring sensors 502 may be one or more sensors including RADAR, LIDAR. IR, imaging, EEG-based, and the like.

The contactless biomonitoring sensors 502 can determine how many passengers are going to board the vehicle 500 by detecting one or more different heart activities and respiration patterns, for example. Based on the number of people the contactless biomonitoring sensors 502 have detected, a number of seats 508 corresponding to the number of people waiting to board may be deployed before anyone boards.

As a passenger 504 approaches a doorway 506, the vehicle 500 may recognize the location of the passenger 504 at the location of the doorway 506 and may check for accompanying items. The contactless biomonitoring sensors 502 may determine that the passenger 504 has an accompanying item based on recognition of the shape of the object and/or recognition of the passenger's gait pattern that is indicative of a person with an accompanying object. For example, the passenger 504 may have a wheelchair for an accompanying object. The contactless biomonitoring sensors 502 may recognize the shape of the wheelchair and/or may notice that a gait pattern could not be detected because the passenger 504 is on a wheelchair. The vehicle 500 may now prepare the seating to accommodate the passenger 504 by retracting seats to make room for the passenger 504.

Taking a vehicle action before passengers enter the vehicle 500 has the added benefit of preventing illnesses from spreading to other passengers of the vehicle 500. The exterior contactless biomonitoring sensors 502 may detect that a passenger 504 is infected with a respiration virus by determining the respiration as well as the temperature of the passenger. If the passenger 504 is determined to have a fever in conjunction with poor breathing, the vehicle 500 may prevent the passenger 504 from boarding the vehicle 500 to prevent the spread of the illness. The vehicle 500 may also issue a notice to the passenger 504 that the passenger 504 has been denied entry to the vehicle 500 due to the passenger's illness.

Taking a vehicle action before passengers enter the vehicle 500 also has the added benefit of determining a passenger's authorization to enter the vehicle 500. The exterior contactless biomonitoring sensors 502 may include gait and/or EEG-based sensors to authenticate a passenger 504. Gait sensors may include sensors such as LIDAR and RADAR. EEG-based sensors include sensors such as neurobiomonitoring (NBM) coils. The vehicle 500 may be remotely connected to a database of passenger profiles.

The contactless biomonitoring sensors 502 may collect gait data and/or brainwave data to compare to the database of passenger profiles to determine if the passenger 504 is authorized to board the vehicle 500. Gait data may be the passenger's gait as the passenger approached the vehicle 500. Brainwave data may be EEG data in response to a stimulus to the passenger from the vehicle 500 such as, for example, an image displayed on a screen of the vehicle. The contactless biomonitoring sensors 502 may detect and analyze the brainwaves of an approaching passenger to identify the passenger 504. The passenger 504 could have a predetermined password such as a repetitive task, word, or phrase to think of to trigger a specific brainwave response that the vehicle 500 may then process to identify the passenger 504. In another embodiment, the exterior of the vehicle 500 may have a display that may present one or more graphics to be viewed by the passenger 504 to trigger a specific brainwave response (much like a password) that the vehicle 500 may process to identify the passenger 504.

If the passenger 504 is not authorized to enter, the vehicle 500 may prevent the passenger 504 from boarding the vehicle 500. The vehicle 500 may also issue a notice to the passenger 504 that the passenger 504 has been denied entry to the vehicle 500 due to the passenger's illness.

Although an autonomous rideshare or public transportation vehicle was referenced for purposes of illustration, any vehicle could implement embodiments of the present disclosure. For example, vehicles that are manually driven may include embodiments of the present disclosure, and drivers may be used to confirm the conditions that embodiments have detected before taking action.

It should now be understood that disclosed herein are embodiments directed to systems, methods, and vehicles for passenger transportation and health monitoring. Embodiments may determine a location of one or more passengers within an area of a vehicle and identify a health condition, action, and/or accompanying item of the one or more passengers. Based on the determined location and the identified health condition, action, and/or accompanying item, the vehicle may be directed to perform a vehicle action.

One or more contactless biomonitoring sensors determine the location and identifies the health condition, action, and/or accompanying item. A contactless biomonitoring sensor is one or more sensors that may be used to measure biometrics and/or vital signs without contact with a user. The contactless biomonitoring sensor can measure multiple individuals at once

It is also noted that recitations herein of “at least one” component, element, etc., should not be used to create an inference that the alternative use of the articles “a” or “an” should be limited to a single component, element, etc.

It is noted that recitations herein of a component of the present disclosure being “configured” or “programmed” in a particular way, to embody a particular property, or to function in a particular manner, are structural recitations, as opposed to recitations of intended use. More specifically, the references herein to the manner in which a component is “configured” or “programmed” denotes an existing physical condition of the component and, as such, is to be taken as a definite recitation of the structural characteristics of the component.

It is noted that one or more of the following claims utilize the term “wherein” as a transitional phrase. For the purposes of defining the present invention, it is noted that this term is introduced in the claims as an open-ended transitional phrase that is used to introduce a recitation of a series of characteristics of the structure and should be interpreted in like manner as the more commonly used open-ended preamble term “comprising.”

Having described the subject matter of the present disclosure in detail and by reference to specific embodiments thereof, it is noted that the various details disclosed herein should not be taken to imply that these details relate to elements that are essential components of the various embodiments described herein, even in cases where a particular element is illustrated in each of the drawings that accompany the present description. Further, it will be apparent that modifications and variations are possible without departing from the scope of the present disclosure, including, but not limited to, embodiments defined in the appended claims. More specifically, although some aspects of the present disclosure are identified herein as preferred or particularly advantageous, it is contemplated that the present disclosure is not necessarily limited to these aspects.

What is claimed is:

1. A method for passenger transportation and health monitoring, the method comprising:
determining, with a contactless biomonitoring sensor, a location of one or more passengers within a monitoring area of a vehicle;
generating, with the contactless biomonitoring sensor, a health profile of one or more passengers;
identifying, with a processor, a health condition, an action, an accompanying item, or combinations thereof of one or more passengers based on the health profile corresponding to the one or more passengers;
determining, with the processor, a vehicle action based on the health condition, the action, the accompanying item, or combinations thereof; and
directing, with the processor, the vehicle to perform the vehicle action.

2. The method of claim 1, further comprising:
prompting a driver of the vehicle to confirm the vehicle action prior to directing the vehicle to perform the vehicle action.

3. The method of claim 1, wherein the health profile includes a set of biometric data, a set of vital signs, or combinations thereof.

4. The method of claim 3, wherein the set of biometric data includes brainwaves, gait patterns, or combinations thereof.

5. The method of claim 3, wherein the set of vital signs includes heart activity, respiration rate, body temperature, blood oxygenation, or combinations thereof.

6. The method of claim 1, wherein generating the health profile of one or more passengers comprises:
gathering, with the contactless biomonitoring sensor, a set of health data from within the monitoring area of the vehicle;
segmenting the set of health data into health profiles based on the location of one or more passengers; and
assigning the health profiles to the one or more passengers at the location corresponding to the one or more passengers.

7. The method of claim 1, wherein the contactless biomonitoring sensor is a LIDAR sensor, a RADAR sensor, an ultrasound sensor, an EEG-based sensor, an IR sensor, an imaging sensor, or combinations thereof.

8. The method of claim 1, wherein the vehicle action is adjusting a seat, adjusting a storage compartment, driving to a destination, making a phone call, evacuating the vehicle, adjusting a comfort system, adjusting a door, issuing a notice, or combinations thereof.

9. A system for passenger transportation and health monitoring, the system comprising:
a processor;
a contactless biomonitoring sensor communicatively coupled to the processor;
a memory module communicatively coupled to the processor; and
machine-readable instructions stored in the memory module that, when executed by the processor, cause the processor to perform at least the following:
determine, with the contactless biomonitoring sensor, a location of one or more passengers within a monitoring area of a vehicle;
generate, with the contactless biomonitoring sensor, a health profile of one or more passengers;
identify, with the processor, a health condition, an action, an accompanying item, or combinations thereof of one or more passengers based on the health profile corresponding to the one or more passengers;
determine, with the processor, a vehicle action based on the health condition, the action, the accompanying item, or combinations thereof; and
direct, with the processor, the vehicle to perform the vehicle action.

10. The system of claim 9, wherein the machine-readable instructions further cause the processor to:
prompt a driver of the vehicle to confirm the vehicle action prior to directing the vehicle to perform the vehicle action.

11. The system of claim 9, wherein the health profile includes a set of biometric data, a set of vital signs, or combinations thereof.

12. The system of claim 11, wherein the set of biometric data includes brainwaves, gait patterns, or combinations thereof.

13. The system of claim 11, wherein the set of vital signs includes heart activity, respiration rate, body temperature, blood oxygenation, or combinations thereof.

14. The system of claim 9, wherein generating the health profile of one or more passengers comprises:
gathering, with the contactless biomonitoring sensor, a set of health data from within the monitoring area of the vehicle;
segmenting the set of health data into health profiles based on the location of one or more passengers; and
assigning the health profiles to the one or more passengers at the location corresponding to the one or more passengers.

15. The system of claim 9, wherein the contactless biomonitoring sensor is a LIDAR sensor, a RADAR sensor, an ultrasound sensor, an EEG-based sensor, an IR sensor, an imaging sensor, or combinations thereof.

16. The system of claim 9, wherein the vehicle action is adjusting a seat, adjusting a storage compartment, driving to a destination, making a phone call, evacuating the vehicle, adjusting a comfort system, adjusting a door, issuing a notice, or combinations thereof.

17. A vehicle for passenger transportation and health monitoring, the vehicle comprising:
a processor;
a contactless biomonitoring sensor communicatively coupled to the processor;
a memory module communicatively coupled to the processor; and
machine-readable instructions stored in the memory module that, when executed by the processor, cause the processor to perform at least the following:
determine, with the contactless biomonitoring sensor, a location of one or more passengers within a monitoring area of the vehicle;
generate, with the contactless biomonitoring sensor, a health profile of one or more passengers;
identify, with the processor, a health condition, an action, an accompanying item, or combinations thereof of one or more passengers based on the health profile corresponding to the one or more passengers;
determine, with the processor, a vehicle action based on the health condition, the action, the accompanying item, or combinations thereof; and
direct, with the processor, the vehicle to perform the vehicle action.

18. The vehicle of claim 17, wherein generating the health profile of one or more passengers comprises:
gathering, with the contactless biomonitoring sensor, a set of health data from within the monitoring area of the vehicle;
segmenting the set of health data into health profiles based on the location of one or more passengers; and
assigning the health profiles to the one or more passengers at the location corresponding to the one or more passengers.

19. The vehicle of claim 17, wherein the contactless biomonitoring sensor is a LIDAR sensor, a RADAR sensor, an ultrasound sensor, an EEG-based sensor, an IR sensor, an imaging sensor, or combinations thereof.

20. The vehicle of claim 17, wherein the vehicle action is adjusting a seat, adjusting a storage compartment, driving to a destination, making a phone call, evacuating the vehicle, adjusting a comfort system, adjusting a door, issuing a notice, or combinations thereof.
